(19) United States
(12) Patent Application Publication
(10) Pub. No.: US 2005/0031598 A1
(43) Pub. Date: Feb. 10, 2005

(54) Engineering three-dimensional tissue structures using differentiating embryonic stem cells

(76) Inventors: Shulamit Levenberg, Brighton, MA (US); Ngan F. Huang, Berkeley, CA (US); Erin B. Lavik, New Haven, CT (US); Joseph Itskovitz-Eldor, Haifa (IL); Robert Langer, Newton, MA (US)

Correspondence Address: Patent Department;Choate, Hall & Stewart, 53 State Street, Exchange Place, Boston, MA, 02109, US

(21) Appl. No.: 10/731,672

(22) Filed: Dec. 9, 2003

Related U.S. Application Data
(60) Provisional application No. 60/432,228, filed on Dec. 10, 2002.
(60) Provisional application No. 60/443,926, filed on Jan. 31, 2003.

(51) Int. Cl. C12N 5/08

(52) U.S. Cl. 424/93.7; 435/366

(57) ABSTRACT

A method of producing a tissue engineering construct. The method includes providing a population of embryonic stem cells, seeding the embryonic stem cells on a cell support matrix, and exposing the embryonic stem cells to at least one agent selected to promote differentiation of the stem cells along a predetermined cell lineage or into a specific cell type. The step of exposing may be performed before or after the step of seeding.

This application claims the priority of Provisional Patent Application No. 60/432,228, filed Dec. 10, 2002 and Provisional Patent Application No. 60/443,926, filed Jan. 31, 2003.

### FIELD OF THE INVENTION

This invention pertains to the production of three-dimensional tissue structures using differentiating embryonic stem cells.

### BACKGROUND OF THE INVENTION

Embryonic stem (ES) cells, including human ES (hES) cells, are a promising source for cell transplantation due to their unique ability to give rise to all somatic cell lineages when they undergo differentiation^{1-3,4}. Differentiation of ES can be induced by removing the cells from their feeder layer and growing them in suspension, resulting in cellular aggregation and formation of embryoid bodies (EBs), in which successive differentiation steps occur⁵. Several studies have shown that chemical cues provided directly by growth factors or indirectly by feeder cells can induce ES cell differentiation towards specific lineages⁶⁻⁹. However, none of these studies succeeded in controlling differentiation of the ES cells to form complex tissues. In some cell types, physical cues including surface interactions, shear stress and mechanical strain have induced differentiation¹⁰⁻¹³.

Thus, it is desirable to develop methods of promoting differentiation of ES cells into three-dimensional tissue structures.

### SUMMARY OF THE INVENTION

In one aspect, the invention provides a tissue engineering construct including embryonic stem cells, a three-dimensional cell support matrix that is resistant to contractile forces exerted by the stem cells, and at least one growth factor selected to promote differentiation of the stem cells along a predetermined cell lineage or into a specific cell type. The stem cells may be mammalian embryonic stem cells, for example, human embryonic stem cells. The cell support matrix may include a poly(lactic acid)-poly(lactic acid-co-glycolic acid) mixture, for example a 50/50 mixture of poly(L-lactic acid) and poly(lactic acid-co-glycolic acid).

A cross-sectional area of the matrix may be reduced by not more than 50% under a contractile force exerted by the embryonic stem cells, for example, not more than 40%, 30%, 20%, 10%, or 1%. The cell support matrix may further include a coating including an agent that promotes cell adhesion, for example, fibronectin, integrins, or oligonucleotides that promote cell adhesion. The cell support matrix may be biodegradable or non-biodegradable.

The tissue engineering construct may further include one or more biomolecules, small molecules, or bioactive agents disposed within the cell support matrix. The tissue engineering construct may further include a gel that coats internal and external surfaces of cell support matrix. Exemplary gels include collagen gel, alginate, agar, and Growth Factor Reduced Matrigel. The gel may further include one or more of laminin, fibrin, fibronectin, proteoglycans, glycoproteins, glycosaminoglycans, chemotactic agents, or growth factors, for example, cytokines, eicosanoids, or differentiation factors.

In another aspect, the invention provides a method of producing a tissue engineering construct. The method includes providing a population of embryonic stem cells, seeding the embryonic stem cells on a cell support matrix, and exposing this embryonic stem cells to at least one agent selected to promote differentiation of the stem cells along a predetermined lineage or into a specific cell type. The step of exposing may be performed before or after the step of seeding and may be performed in a serum-free medium. The cell support matrix may be three-dimensional and may be coated with an agent that promotes cell adhesion. The embryonic stem cells may be disposed within a gel, and seeding the embryonic stem cells on the cell support matrix may include disposing the gel on internal and external surfaces of the cell support matrix.

The agent may be a growth factor, a mechanical force, an electrical voltage, a bioactive agent, a biomolecule, a small molecule, or some combination of these. The mechanical force may include a hoop stress, a shear stress, a hydrostatic stress, a compressive stress, a tensile stress, or any combination of these. The embryonic stem cells may be cultured in the presence of a growth factor as part of the step of providing.

### Definitions

“Biomolecules”: The term “biomolecules”, as used herein, refers to molecules (e.g., proteins, amino acids, peptides, polynucleotides, nucleotides, carbohydrates, sugars, lipids, nucleoproteins, glycoproteins, lipoproteins, steroids, etc.) whether naturally-occurring or artificially created (e.g., by synthetic or recombinant methods) that are commonly found in cells and tissues. Specific classes of biomolecules include, but are not limited to, enzymes, receptors, neurotransmitters, hormones, cytokines, cell response modifiers such as growth factors and chemotactic factors, antibodies, vaccines, haptens, toxins, interferons, ribozymes, anti-sense agents, plasmids, DNA, and RNA.

“Biocompatible”: The term “biocompatible”, as used herein is intended to describe materials that do not elicit an undesirable detrimental response in vivo.

“Biodegradable”: As used herein, “biodegradable” polymers are polymers that degrade fully (i.e., down to monomeric species) under physiological or endosomal conditions. In preferred embodiments, the polymers and polymer biodegradation byproducts are biocompatible. Biodegradable polymers are not necessarily hydrolytically degradable and may require enzymatic action to fully degrade.

“Growth Factors”: As used herein, “growth factors” are chemicals that regulate cellular metabolic processes, including but not limited to differentiation, proliferation, synthesis of various cellular products, and other metabolic activities. Growth factors may include several families of, chemicals, including but not limited to cytokines, eicosanoids, and differentiation factors.

“Polynucleotide”, “nucleic acid”, or “oligonucleotide”: The terms “polynucleotide”, “nucleic acid”, or “oligonucleotide” refer to a polymer of nucleotides. The terms “polynucleotide”, “nucleic acid”, and “oligonucleotide”, may be used interchangeably. Typically, a polynucleotide comprises at least three nucleotides. DNAs and RNAs are polynucleotides. The polymer may include natural nucleosides (i.e., adenosine, thymidine, guanosine, cytidine, uridine, deoxyadenosine, deoxythymidine, deoxyguanosine, and deoxycytidine), nucleoside analogs (e.g., 2-aminoadenosine, 2-thiothymidine, inosine, pyrrolo-pyrimidine, 3-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, O(6)-methylguanine, and 2-thiocytidine), chemically modified bases, biologically modified bases (e.g., methylated bases), intercalated bases, modified sugars (e.g., 2′-fluororibose, ribose, 2′-deoxyribose, arabinose, and hexose), or modified phosphate groups (e.g., phosphorothioates and 5′-N-phosphoramidite linkages).

“Polypeptide”, “peptide”, or “protein”: According to the present invention, a “polypeptide”, “peptide”, or “protein” comprises a string of at least three amino acids linked together by peptide bonds. The terms “polypeptide”, “peptide”, and “protein”, may be used interchangeably. Peptide may refer to an individual peptide or a collection of peptides. Inventive peptides preferably contain only natural amino acids, although non-natural amino acids (i.e., compounds that do not occur in nature but that can be incorporated into a polypeptide chain; see, for example, http://www.cco.caltech.edu/˜dadgrp/Unnatstruct.gif, which displays structures of non-natural amino acids that have been successfully incorporated into functional ion channels) and/or amino acid analogs as are known in the art may alternatively be employed. Also, one or more of the amino acids in an inventive peptide may be modified, for example, by the addition of a chemical entity such as a carbohydrate group, a phosphate group, a farnesyl group, an isofarnesyl group, a fatty acid group, a linker for conjugation, functionalization, or other modification, etc. In a preferred embodiment, the modifications of the peptide lead to a more stable peptide (e.g., greater half-life in vivo). These modifications may include cyclization of the peptide, the incorporation of D-amino acids, etc. None of the modifications should substantially interfere with the desired biological activity of the peptide.

“Polysaccharide”, “carbohydrate” or “oligosaccharide”: The terms “polysaccharide”, “carbohydrate”, or “oligosaccharide” refer to a polymer of sugars. The terms “polysaccharide”, “carbohydrate”, and “oligosaccharide”, may be used interchangeably. Typically, a polysaccharide comprises at least three sugars. The polymer may include natural sugars (e.g., glucose, fructose, galactose, mannose, arabinose, ribose, and xylose) and/or modified sugars (e.g., 2′-fluororibose, 2′-deoxyribose, and hexose).

“Small molecule”: As used herein, the term “small molecule” is used to refer to molecules, whether naturally-occurring or artificially created (e.g., via chemical synthesis), that have a relatively low molecular weight. Typically, small molecules are monomeric and have a molecular weight of less than about 1500 g/mol. Preferred small molecules are biologically active in that they produce a local or systemic effect in animals, preferably mammals, more preferably humans. In certain preferred embodiments, the small molecule is a drug. Preferably, though not necessarily, the drug is one that has already been deemed safe and effective for use by the appropriate governmental agency or body. For example, drugs for human use listed by the FDA under 21 C.F.R. §§ 330.5, 331 through 361, and 440 through 460; drugs for veterinary use listed by the FDA under 21 C.F.R. §§ 500 through 589, incorporated herein by reference, are all considered acceptable for use in accordance with the present invention.

“Bioactive agents”: As used herein, “bioactive agents” is used to refer to compounds or entities that alter, inhibit, activate, or otherwise affect biological or chemical events. For example, bioactive agents may include, but are not limited to, anti-AIDS substances, anti-cancer substances, antibiotics, immunosuppressants, anti-viral substances, enzyme inhibitors, neurotoxins, opioids, hypnotics, anti-histamines, lubricants, tranquilizers, anti-convulsants, muscle relaxants and anti-Parkinson substances, anti-spasmodics and muscle contractants including channel blockers, miotics and anti-cholinergics, anti-glaucoma compounds, anti-parasite and/or anti-protozoal compounds, modulators of cell-extracellular matrix interactions including cell growth inhibitors and anti-adhesion molecules, vasodilating agents, inhibitors of DNA, RNA or protein synthesis, anti-hypertensives, analgesics, anti-pyretics, steroidal and non-steroidal anti-inflammatory agents, anti-angiogenic factors, anti-secretory factors, anticoagulants and/or antithrombotic agents, local anesthetics, ophthalmics, prostaglandins, anti-depressants, anti-psychotic substances, anti-emetics, and imaging agents. In certain embodiments, the bioactive agent is a drug.

A more complete listing of bioactive agents and specific drugs suitable for use in the present invention may be found in “Pharmaceutical Substances: Syntheses, Patents, Applications” by Axel Kleemann and Jurgen Engel, Thieme Medical Publishing, 1999; the “Merck Index: An Encyclopedia of Chemicals, Drugs, and Biologicals”, Edited by Susan Budavari et al., CRC Press, 1996, and the United States Pharmacopeia-25/National Formulary-20, published by the United States Pharmcopeial Convention, Inc., Rockville Md., 2001, all of which are incorporated herein by reference.

“Tissue”: as used herein, the term “tissue” refers to a collection of cells of one or more types combined to perform a specific function, and any extracellular matrix surrounding the cells.

### BRIEF DESCRIPTION OF THE DRAWING

The invention is described with reference to the several figures of the drawing, in which,

FIG. 1 includes light micrographs of control tissues stained with antibodies to their characteristic proteins or histological stains to determine specificity and optimal dilution. (A and B) nestin, mouse embryonic brain (embryonic day 17); (C) β_{III}-tubulin, mouse subcutaneous; (D) cytokeratin-7, human lung; (E) insulin, human pancreas; (F) β_{III}-tubulin, mouse brain; (G) vimentin, human tonsil; (H) smooth muscle actin, human tonsil; (I) CD34, human tonsil; (J) CD31, human tonsil; (K) albumin, liver; (L) α-feto-protein (AFP), adult liver; (M) safranin-O, fibrous cartilage.

FIG. 2A includes light micrographs of differentiating hES cells (EB day 8) mixed with matrigel and grown for two weeks in the presence of transforming growth factor beta (TGF), activin-A (ACT), retinoic acid (RA) insulin growth factor (IGF) or no growth factor (CON). Left panel: dark field images of the “spheres” formed (Scale bars=1 mm). Middle and right panels: histological sections of the samples stained with H&E. Bottom: histochemical and immunostaining of cross sections of the “spheres” formed in matrigel with Safranin-O (SafO), anti-AFP and anti-nestin antibodies (scale bars=100 μm).

FIGS. 2B-D illustrate the results of mechanical testing of PLGA/PLA scaffolds with or without matrigel. Tensile strength tests (B) and compression tests (C) results are summarized in comparison to matrigel (D).

FIG. 3 is a photograph of a gel showing the products of RT-PCR using primers for ultra-high sulfur keratin (keratin), neurofilament heavy chain (NFH), cartilage matrix protein (CMP), α-feto-protein (AFP), PDX-1, and GAPDH on RNA isolated from eight-day-old embryoid bodies (EBs) trypsinized, seeded on fibronectin-coated plates, and grown for 2 weeks in the presence of transforming growth factor β (TGF), activin-A (ACT), retinoic acid (RA), insulin-like growth factor (IGF), vascular endothelial growth factor (VEGF), or control medium (CON).

FIG. 4 includes light micrographs of 5-μm-thick sections taken from hEBs (day 8), incubated for additional 2 weeks with control medium (CON) or medium supplemented with retinoic acid (RA), or insulin-like growth factor (IGF), and stained with antibodies against human cytokeratin, α-feto-protein, and nestin (scale bars=200 μm.)

FIGS. 5A-D are scanning electron micrographs of PLLA/PLGA scaffolds without (A) and with (B-D) differentiating hES cells, showing the attachment of the cells to the scaffolds in different magnifications (scale bars: A,B=1 mm, C=50 μm, D=200 μm).

FIGS. 5E-H include light micrographs of PLLA/PLGA scaffolds stained with hematoxylin and eosin (H&E) stain. hES cells were seeded onto the scaffold by (E, G) seeding the cells onto the scaffold with matrigel or (F, H) coating the scaffold with fibronectin (scale bars=50 μm).

FIGS. 5I-K illustrate the proliferation of hES cells on PLLA/PLGA scaffolds after two weeks of culture, incubation with BrdUrd, and staining with anti-BrdUrd antibodies (brown) [(I) Low (×100) and (J-K) high (×1000) magnifications] (scale bars=50 μm).

FIG. 6 includes micrographs of undifferentiated (undiff) or differentiating hES cells [embryoid body (EB) day 8] (diff), mixed with matrigel, seeded on PLLA/PLGA scaffolds, cultured for 2 weeks, and stained with H&E or with antibodies against human α-feto-protein (AFP), nestin, or β_{III}-tubulin (Original magnification, ×200, except when indicated ×400).

FIG. 7A includes light micrographs of hES cell-scaffold constructs grown for two weeks in control medium (CON) or in the presence of insulin growth factor (IGF) or retinoic acid (RA), sectioned and stained with anti-cytokeratin antibodies (red), anti-vimentin antibodies (green), and DAPI for nuclear staining (blue) (scale bars=100 μm).

FIG. 7B includes light micrographs of hES cell-scaffold constructs grown for two weeks in control medium (CON) or in the presence of transforming growth factor-β (TGFβ) or retinoic acid (RA), sectioned and stained with trichrome for collagen (blue) (scale bars=100 μm).

FIG. 7C is a graph comparing lumen diameters of tubulocystic structures lined by cytokeratin-positive epithelium in constructs grown for two weeks in control medium or in the presence of IGF or RA

FIG. 7D is a graph illustrating the percentage of area positively stained (percentage of positive staining) with anti-cytokeratin antibody within tissue sections from samples obtained in two different experiments performed in duplicates and sections of normal human lung tissue (Epithelia) (bar indicates mean value +/− SD).

FIG. 8A illustrates immunostaining of tissue sections taken from hES constructs incubated for two weeks with control medium (CON) or medium supplemented with TGF-β (TGF), activin-A (ACT), retinoic acid (RA), insulin growth factor (IGF) or a combination of TGF-β and activin-A (TGF/ACT) and stained with Safranin O (Saf O) or with antibodies against human AFP, albumin, nestin, β_{III}-tubulin and S-100 (scale bars=50 μm).

FIG. 8B is a graph illustrating the percentage of area positively stained (percentage of positive staining) with the indicated stains or antibodies within tissue sections from samples obtained in three different experiments performed in duplicate (bar indicates mean value +/− SD).

FIG. 9A is a photograph of a gel showing the results of RT-PCR using primers for ultra high sulfur keratin (keratin), neurofilament heavy chain (NFH), cartilage matrix protein (CMP), alpha feto protein (AFP), PDX-1, CD34 and GAPDH on RNA isolated from tissue constructs grown for two weeks in the presence of TGF-β (TGF), activin-A (ACT), RA, IGF, or control medium (CON).

FIG. 9B is a schematic representation of the effects of various growth factors on the expression of tissue-specific genes in 3D constructs based on semi quantitative analysis of gene expression (+=low expression; ++++=highest expression).

FIG. 10A is a series of light micrographs of differentiating hES cells (EB day 8) seeded on PLLA/PLGA scaffolds with matrigel (s+m) or after coating the scaffold with fibronectin (s+fn), incubated in a control medium (CON) or medium supplemented with TGF-β (TGF), activin-A (ACT), RA, or IGF, and, following two weeks of incubation, fixed, sectioned and immunostained using anti-CD31, anti-CD34, or anti-smooth muscle actin (SMA) antibodies (scale bar=50 μm).

FIG. 10B is a graph illustrating the percentage of positive staining (area of antibody-positive cells within the tissue sections) in the constructs discussed in FIG. 10A (values reflect mean values (±SD) of 5 different sample sections).

FIG. 11 includes light micrographs of two-week old hES-scaffold constructs implanted into SCID mice and stained with H&E or with antibodies against human CD31, cytokeratin, AFP, or β_{III}-tubulin (scale bar=50 μm).

FIG. 12A includes micrographs of sample sections (after 2 weeks) of PLLA/PGLA scaffolds seeded with differentiating human embryonic stem (hES) cells [embryoid body (EB) day 8] and matrigel, stained with antibodies against human desmin, myogenin, and insulin. Desmin-positive cells were found in the constructs, with some elongated cells. No myogenin cells were found in the constructs. Insulin-positive cells were extremely rare.

FIG. 12B includes micrographs of two-week-old constructs implanted subcutaneously in the dorsal region of severe combined immunodeficient (SCID) mice and stained with antibodies against Tra 1-60 and SSEA-4 after 14 days in vivo, with undifferentiated hES cells seeded on scaffolds for 1 day (ES 1 day) serving as a control.

### DETAILED DESCRIPTION

In one embodiment, the invention is a method of producing a tissue engineering construct. A population of hES cells is seeded on a support matrix before or after being exposed to an agent that stimulates a desired differentiation path. The support matrix should have a modulus sufficiently high to resist collapse under the contractile forces exerted by the cells.

We have unexpectedly discovered that combining the appropriate chemical and physical cues creates a supportive environment to direct differentiation and organization of hES cells into three dimensional (3D) tissue structures. We have created a series of 3D culture conditions using matrigel and biodegradable scaffolds and found that the physical cues provided by the biodegradable scaffolds promoted the formation of tissue-like structures. Specifically, polymer scaffolds designed to resist contraction under the compressive stress exerted by the cells promoted proliferation, differentiation and organization of hES cells into 3D structures. Furthermore, variation of growth factor conditions induced formation of human tissue-like structures including cartilage, liver, and neural tissues. Finally, hES cells cultured on polymer scaffolds organized into an endothelial tube-network, vascularizing the tissue in vitro. Thus, physical environment is an influential parameter in hES cell differentiation into 3D tissues.

The cells may be cultured in the absence of LIF and bFGF to induce the formation of embryoid bodies and then trypsinized. The cells may be directly seeded onto a three-dimensional matrix or combined with a gel for seeding. An exemplary gel is Growth-Factor Reduced Matrigel™ (matrigel), available from Becton-Dickinson. Unmodified matrigel is a solubilized basement membrane matrix extracted from the EHS mouse tumor (Kleinman, H. K., et al., Biochem. 25:312, 1986). The primary components of the matrix are laminin, collagen I, entactin, and heparan sulfate proteoglycan (perlecan) (Vukicevic, S., et al., Exp. Cell Res. 202:1, 1992). Growth Factor-Reduced Matrigel is produced by removing most of the growth factors from the matrix (see Taub, et al., Proc. Natl. Acad. Sci. USA, (1990);87(10):4002-6). Alternatively, the gel may be a collagen I gel. Additional gels that may be used with the invention include but are not limited to alginate, fibrin, agar, and collagen IV.

If a gel is used, it may also include other extracellular matrix components, such as glycosaminoglycans, fibrin, fibronectin, proteoglycans, and glycoproteins. The gel may also include basement membrane components such as collagen IV and laminin. In one embodiment, extracellular matrix components found in tissues containing the same type of cells as the stem cells are being differentiated into may be incorporated into the gels. Enzymes such as proteinases and collagenases may be added to the gel, as may cell response modifiers such as growth factors and chemotactic agents.

The gel will be absorbed onto the interior and exterior surfaces of the matrix and may fill some of the pores of a porous matrix. Capillary forces will retain the gel on the matrix before hardening, or the gel may be allowed to harden on the matrix to become more self-supporting.

The three-dimensional matrix is preferably sufficiently stiff that it does not collapse under the contractile forces exerted by the differentiating cells. The mean asymptotic force per cell (F_{cell}) has been calculated to be approximately 3 nN for fibroblasts independent of scaffold stiffness³⁸. While it is a broad assumption, if one uses that value to represent the force (σ) an average cell would exert then the following would hold:

$$σ = \frac{{F}_{cell} \times numberofcells}{Areaofcells}$$

That being true, one can estimate the number of cells in a cross sectional area by dividing the cross sectional area (Areaofcells) by the cross sectional area of a single cell (A_{cell}). The above equation can be re-expressed as the following:

$$σ = \frac{{F}_{cell}}{{A}_{cell}}$$

If one assumes the diameter of a cell in cross section is approximately 6 μm, then A_{cell} is approximately (assuming a circular cross section) 28 μm. Substituting these known values into the above equation gives the following result: cells exert a stress of approximately 110 Pa on a scaffold. This is a very general, broad estimate.

In one embodiment, the embryonic stem cells are able to maintain three dimensional structures after being seeded on the matrix, and the cross-sectional area of the matrix is not reduced by more than 50%, for example, less than 40% with respect to an unseeded matrix, as the cells perform various cell functions (e.g., metabolic functions, proliferation, differentiation). In some embodiments, the cross-sectional area is reduced by less than 30% or even less, for example, less than 20%, less than 10%, or less than 1% under the mechanical forces exerted by the seeded cells. One skilled in the art will understand how to select polymers and adjust their moduli, for example, by controlling the molecular weight and cross-link density, to optimize the amount of contraction.

In some embodiments, the matrix may be formed with a microstructure similar to that of the extracellular matrix that is being replaced. The molecular weight, tacticity, and cross-link density of the matrix may also be regulated to control both the mechanical properties of the matrix and the degradation rate (for degradable scaffolds). The mechanical properties may also be optimized to mimic those of the tissue at the implant site. The shape and size of the final implant should be adapted for the implant site and tissue type. The matrix may serve simply as a delivery vehicle for the stem cells or may provide a structural or mechanical function. The matrix may be formed in any shape, for example, as particles, a sponge, a tube, a sphere, a strand, a coiled strand, a capillary network, a film, a fiber, a mesh, or a sheet.

The porosity of the matrix may be controlled by a variety of techniques known to those skilled in the art. The minimum pore size and degree of porosity is dictated by the need to provide enough room for the cells and for nutrients to filter through the matrix to the cells. The maximum pore size and porosity is limited by the ability of the matrix to maintain its mechanical stability after seeding. As the porosity is increased, use of polymers having a higher modulus, addition of stiffer polymers as a co-polymer or mixture, or an increase in the cross-link density of the polymer may all be used to increase the stability of the matrix with respect to cellular contraction.

The matrices may be made by any of a variety of techniques known to those skilled in the art. Salt-leaching, porogens, solid-liquid phase separation (sometimes termed freeze-drying), and phase inversion fabrication may all be used to produce porous matrices. Fiber pulling and weaving (see, e.g. Vacanti, et al., (1988) Journal of Pediatric Surgery, 23: 3-9) may be used to produce matrices having more aligned polymer threads. Those skilled in the art will recognize that standard polymer processing techniques may be exploited to create polymer matrices having a variety of porosities and microstructures.

Preferably, the polymer matrix is biodegradable. Suitable biodegradable polymers for use in the practice of the invention are well known in the art and include poly(lactic acid) (PLA), poly(glycolic acid) (PGA) and PLA-PGA co-polymers (PLGA). Additional biodegradable materials include PLA, poly(anhydrides), poly(hydroxy acids), poly(ortho esters), poly(propylfumerates), poly(caprolactones), polyamides, polyamino acids, polyacetals, biodegradable polycyanoacrylates, biodegradable polyurethanes and polysaccharides. Non-biodegradable polymers may also be used as well. Other non-biodegradable, yet biocompatible polymers include polypyrrole, polyanilines, polythiophene, polystyrene, polyesters, non-biodegradable polyurethanes, polyureas, poly(ethylene vinyl acetate), polypropylene, polymethacrylate, polyethylene, polycarbonates, and poly(ethylene oxide). Those skilled in the art will recognize that this is an exemplary, not a comprehensive, list of polymers appropriate for tissue engineering applications.

Co-polymers, mixtures, and adducts of the above polymers may also be used in the practice of the invention. Indeed, co-polymers may be particularly useful for optimizing the mechanical and chemical properties of the matrix. For example, a polymer with a high affinity for stem cells may be combined with a stiffer polymer to produce a matrix having the requisite stiffness to resist collapse. For example, PLA may be combined with poly(caprolactone) or PLGA to form a mixture. Both the choice of polymer and the ratio of polymers in a co-polymer may be adjusted to optimize the stiffness of the matrix.

PLA and PLA/PGA copolymers are particularly useful for forming the biodegradable matrices. The erosion of the polyester matrix is related to the molecular weight and crystallinity of the polymer. The higher molecular weights, e.g., weight average molecular weights of 90,000 or higher, result in polymer matrices which retain their structural integrity for longer periods of time; while lower molecular weights, e.g., weight average molecular weights of 30,000 or less, result in shorter matrix lives. The molecular weight and crystallinity also influence the stiffness of the polymer matrix. The tacticity of the polymer also influences the modulus. Poly(L-lactic acid)(PLLA) is isotactic, increasing the crystallinity of the polymer and the modulus of mixtures containing it. One skilled in the art will recognize that the molecular weight and crystallinity of any of the polymers discussed above may be optimized to control the stiffness of the matrix. Likewise, the proportion of polymers in a co-polymer or mixture may be adjusted to achieve a desired stiffness.

In an exemplary embodiment, a cell response modifier such as a growth factor or a chemotactic agent may be added to the polymer matrix. Such a modifier may be used to promote differentiation of the embryonic stem cells into a desired target cell. Alternatively or in addition, the modifier may be selected to recruit cells to the matrix or to promote or inhibit specific metabolic activities of cells recruited to the matrix. Exemplary growth factors include but are not limited to activin-A (ACT), retinoic acid (RA), epidermal growth factor, bone morphogenetic protein, TGF-β, hepatocyte growth factor, platelet-derived growth factor, TGF-α, IGF-I and II, hematopoietic growth factors, heparin binding growth factor, peptide growth factors, erythropoietin, interleukins, tumor necrosis factors, interferons, colony stimulating factors, fibroblast growth factors, nerve growth factor (NGF) and muscle morphogenic factor (MMF). The particular growth factor employed should be appropriate to the desired cell activity and differentiation path. The regulatory effects of a large family of growth factors are well known to those skilled in the art.

The embryonic stem cells may also be cultured with the growth factors or other cell response modifiers before they are seeded on the polymer matrix. These cells will have already started differentiating before being combined with the polymer. Alternatively, different populations of cells that have been exposed to different cell response modifiers may be seeded on different portions of a three-dimensional polymer scaffold.

Additional bioactive agents, biomolecules, and small molecules may also be added to the polymer matrix or to a culture medium before seeding. For example, addition of fibronectin, integrins, or oligonucleotides that promote cell adhesion, such as RGD, may be added to the polymer matrix. Chemotactic or anti-inflammatory agents may be added to the matrix to influence the behavior of cells in the tissue surrounding an implanted matrix.

The cell-seeded polymer matrix, with or without a gel, may be implanted into any tissue, including connective, muscle, nerve, and organ tissues. The techniques of the invention may be used to form tissues of ectodermal, mesodermal, and endodermal origin. In a preferred embodiment, growth factors are selected that will promote differentiation of the ES cells and formation of a predetermined tissue type. For example, addition of TGF-β to hES cells seeded on three-dimensional matrices induces formation of extracellular matrix characteristic of cartilage tissue. Both activin A and IGF induce ES cells to produce proteins characteristic of developing liver. RA induces hES cells to organize into ectodermal structures similar to neuronal tissue. Exposure of ES cells to bone morphogenetic protein, colony stimulating factors specific to bone, and/or PDGF may promote formation of collagen and other bone ECM proteins.

As they differentiate, the cells will produce chemotactic agents that will recruit cells from surrounding tissue to an implanted cell-seeded matrix. Stem cells implanted with the construct will also migrate out of the matrix. The migration of cells will help integrate the implanted construct into the surrounding tissue. Endothelial cells will migrate out of the surrounding blood vessels and develop vasculature within the implanted matrix, providing nutrition to the differentiating cells.

The stem cells express genes and produce proteins characteristic of the target cells well before they are fully differentiated. Thus, stem cells exposed to activin A or IGF express liver specific genes before they fully differentiate into hepatocytes and other cells found in liver. Indeed, not all the stem cells in a population of stem cells exposed to a specific cell response modifier will differentiate the same way. For example, some of the cells exposed to activin A or IGF will express neuronal markers or endothelial markers. These cells can help develop a nervous network and vasculature for the developing liver tissue.

Furthermore, the mechanical interactions of cells and their extracellular matrix influence cellular processes. To further promote differentiation along a desired path, exogenous mechanical forces may be used as a cell response modifier to mimic the mechanical forces exerted by tissues. For example, endothelial cells are exposed to shear forces as blood flows through arteries and veins. Muscle, because it is anchored to bones at least at its ends, is exposed to both uniform and non-uniform tensile stresses. Bone is subjected to compressive and bending stresses during normal locomotion. Organ tissues are exposed to hydrostatic stresses and other compressive stresses. Imposition of mechanical forces on cell-seeded matrices in vitro will influence the production of actin by the seeded stem cells, in turn influencing the degree and type of metabolic activity of the cells and the microstructure of the extracellular matrix they produce.

Similarly, electrical stimulation may be used to influence cell differentiation and metabolism. For example, bone is piezoelectric, and muscle contracts and relaxes in response to electrical signals conducted through nerves. In vitro electrical stimulation imitating the electrical activity of the desired tissue may cause ES cells seeded on a three-dimensional matrix to produce tissue having the electrical characteristics of that tissue.

The shape and microstructure of the polymer matrix and the exogenous forces imposed on the seeded polymer may be optimized for a specific tissue. For example, a medium may be circulated through a seeded tubular substrate in a pulsatile manner (i.e., a hoop stress) to simulate the forces imposed on an artery, or the medium may be used to exert a shear stress on stem cells lining the inside of a tube (Niklason, et al., (1999) Science 284, 489-93; Kaushall, et al., (2001) Nat. Med., 7, 1035-1040). The polymer strands in the matrix may be aligned to mimic the tissue structure of muscle, tendon, or ligament or formed into tubular networks to promote the formation of vasculature.

Even before seeded ES cells are fully differentiated, they can organize themselves into three-dimensional structures characteristic of almost all animal tissue after being exposed to a cell response modifier. Seeded on matrices that can provide a physiologic response to mechanical forces exerted by the stem cells, the stem cells will be able to differentiate and develop under conditions that are more similar to a physiologic environment than a two dimensional petri dish. Indeed, integration of the implant into a tissue site may proceed more quickly or efficiently before the ES cells are terminally differentiated.

### EXAMPLES

Experimental Protocol

Cell Culture

hES cells (H9 clone) were grown on mouse embryonic fibroblasts (Cell Essential, Boston, Mass.) in KnockOut Medium (Gibco-BRL, Gaithersburg, Md.), a modified version of Dulbeco's modified Eagle's medium optimized for ES cells, as described⁵. To induce formation of EBs, hES cell colonies were dissociated with 1 mg/ml collagenase type IV and suspended in differentiation media without LIF and bFGF in Petri dishes⁵.

Scaffold Preparation

The scaffolds consisted of a 50/50 blend of poly(lactic-co-glycolic acid) (Boeringer Ingelheim Resomer 503H, Ingelheim, Germany, Mₙ˜25,000) and poly(L-lactic acid) (Polysciences, Warrington, Pa., Mₙ˜300,000). The sponges were fabricated by a salt-leaching process as described¹⁵. For cell differentiation experiments, the sponges were cut into rectangular pieces of approximately 5×4×1 mm³. Prior to cell seeding, they were sterilized overnight in 70% (vol/vol) ethanol and washed 3 times in PBS.

Mechanical Testing

For tensile testing of the sponge alone, dry sponges were trimmed to 0.4 mm by 5 mm by 11 mm, and tested at a strain rate of 0.05 mm/second until failure using an Instron 5542 apparatus. Compression testing was performed on sponges alone and sponges with Growth Factor-Reduced Matrigel in a parallel plate load cell using the Instron 5542 apparatus. The sponges were porous discs of 17 mm in diameter with a thickness of 0.8 mm. Samples were first precycled one time using to 5% strain at a strain rate of 0.1 mm/mm/second before testing at the same strain rate.

Cell Differentiation on Matrigel and Scaffolds

For seeding in matrigel, 8-9 days-old EBs were trypsinized, and 0.8×10⁶ cells were mixed in 25 μL of a 50% (vol/vol) media and matrigel (growth factor-reduced, BD Biosciences, Bedford, Mass.). EB media was supplemented with the following growth factors: TGF-β1 (2 ng/mL), activin-A (20 ng/mL), and IGF-I (10 ng/mL), (R&D Systems, Minneapolis, Minn.), and RA (300 ng/ml) (Sigma). The mixture was solidified in a 6-well Petri dish at 37° C. and then detached from the dish with sterile blades. 4 mL of each respective EB media was added. For seeding on scaffolds, 0.8×10⁶ cells were seeded into each scaffold using 25 μL of a mixture containing 50% (vol/vol) of Growth Factor-Reduced Matrigel and the respective EB media. After seeding the cells, scaffolds were suspended in 6-well petri dishes in their respective media. For some experiments, scaffolds were soaked in 50 μg/mL of fibronectin (Sigma) for 1 hour and washed in PBS prior to direct cell seeding (without matrigel) in 25 μL of EB media.

Tissue Processing and Immunohistochemical Staining

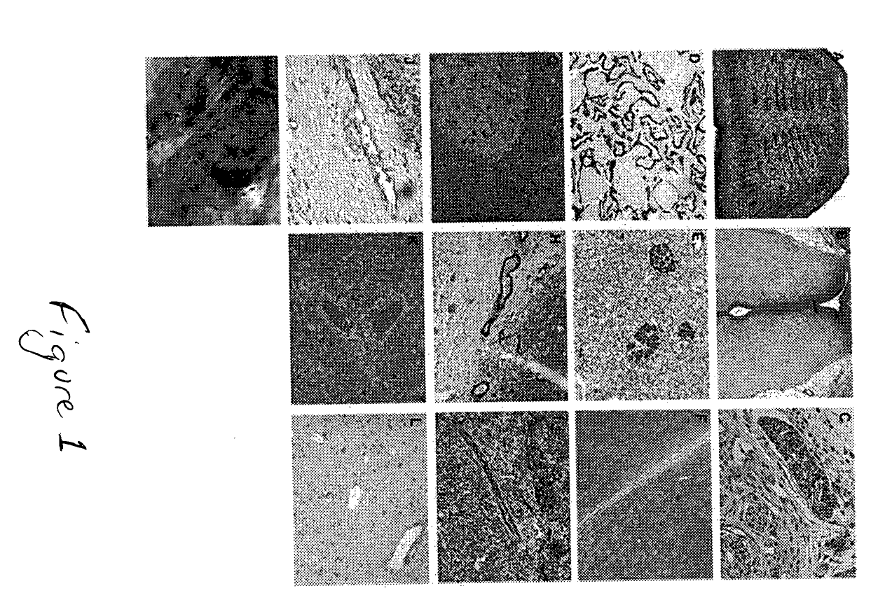

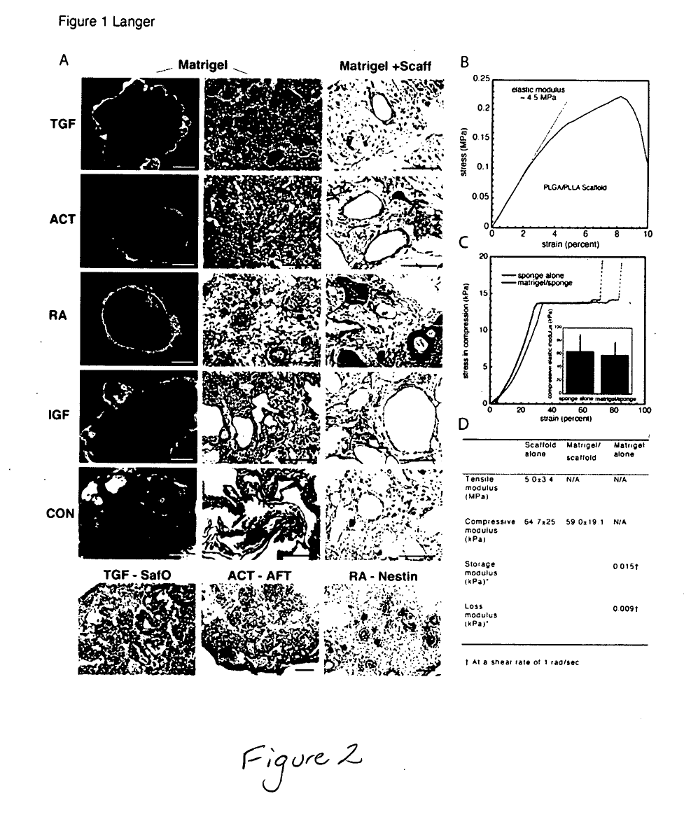

Tissue constructs were fixed for 6 hours in 10% neutral buffered formalin, routinely processed, and embedded in paraffin. 5-μm thick transverse sections were placed on silanized slides for immunohistochemistry or staining with hematoxylin and eosin (H & E), trichrome, or Safranin O. Immunohistochemical staining was carried out using the Biocare Medical Universal HRP-DAB kit (Biocare Medical, Walnut Creek, Calif.) according to the manufacturer's instructions, with prior heat-treatment at 90° C. for 20 minutes in ReVeal buffer (Biocare Medical) for epitope recovery. The primary antibodies were mouse anti-human: desmin (1:150), alpha feto protein (1:2500), cytokeratin 7 (1:25), CD31 (1:20), albumin (1:100), vimentin (1:50), S100 (1:100) (all from Dako), anti-human β_{III}-tubulin (Sigma, 1:500), nestin (Transduction Laboratories, San Diego, Calif., 1:1000), CD34 (Labvision, Fremont, Calif., 1:20), SSEA4 (Hybridoma Bank, University of Iowa, Ames, 1:4), and Tra 1-60 (a gift from Peter Andrews, University of Sheffield, Sheffield, U.K., 1:10). Human and mouse tissues (Daks) were used as controls to ensure antibody specificity (FIG. 1). For proliferation studies, culture medium was incubated with 10 μm of 5′-bromo-2′-deoxyuridine (BrdUrd) (Sigma) for 3 hours before fixation. Tissue sections were stained using mouse anti-BrdUrd antibodies (1:1000).

Comparison of Lumen Diameters of Tubulocystic Structures Lined by Cytokeratin Positive Epithelium

Constructs grown for two weeks in control medium or in the presence of IGF or RA were processed and stained with anti-cytokeratin antibody as described above. Tubulocystic structures were counted and lumen diameters measured and grouped (large>200 μm, medium (Med)>40 μm, small<40 cm, closed and multilayered lumens). The results, the mean values (±SD) of samples obtained in two different experiments performed in duplicate, were recorded as percentages of lumens in each group from total number of lumens in each sample.

Reverse Transcription (RT)-PCR Analysis

Total RNA was isolated by an RNEasy Mini Kit (Qiagen, Chatsworth, Calif.). RT-PCR was carried out using a Qiagen OneStep RT-PCR kit with 10 units RNase inhibitor (Gibco) and 40 ng RNA. Primer sequences, reaction conditions, and cycle numbers were as described^{7,15}. The amplified products were separated on 1.2% agarose gels with ethidium bromide (E-Gel, Invitrogen, Gaithersburg, Mass.). For some gels including RNA amplified using a GADPH primer, semi-quantitative analysis was performed by measuring the mean pixel intensities of each band and normalizing the measured intensity to the mean pixel intensity of the GADPH band.

Transplantation into SCID Mice

Differentiating hES cells that had been grown on scaffolds for 2 weeks in vitro were implanted subcutaneously in the dorsal region of 4-week-old SCID mice (CB.17.SCID, Taconic Farms). Scaffolds implanted without cells were used as controls. Fourteen days after transplantation, the implants were retrieved, fixed overnight in 10% buffered formalin at 4° C., embedded in paraffin, and sectioned for histological examination.

Results

Matrigel Alone does not Provide Sufficient Support for Three-Dimensional hES Cell Differentiation

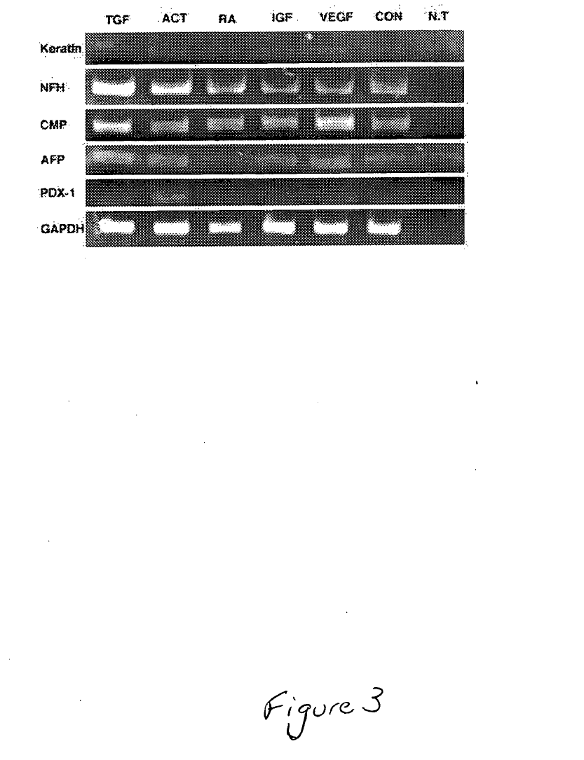

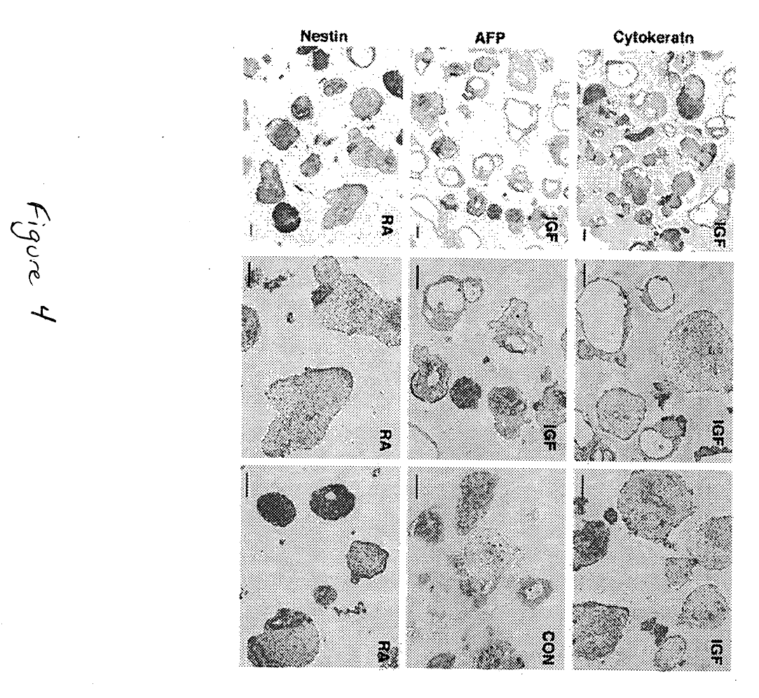

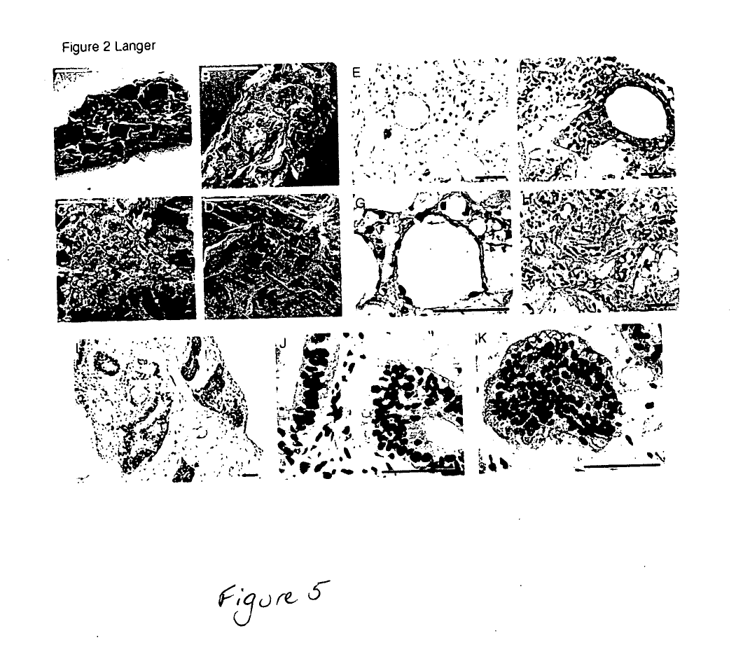

Differentiating hES cells (EBs day 8) were cultured in matrigel, which has been previously shown to support cell organization^{14,15}, in the presence of medium with representative growth factor supplements known to induce ES cell differentiation: retinoic acid (RA), activin-A, transforming growth factor beta (TGF-β), and insulin growth factor (IGF). Initially, the cell-matrigel mixture was shaped into a disc, but after two weeks of culture in suspension, the structure deformed into the shape of a “sphere” suggesting contraction of the matrigel by the cells. Samples treated with either activin-A or RA (and to some extent with TGF-β) formed small, condensed spheres, while samples treated with IGF or control medium with no growth factors were larger and less condensed (FIG. 2A).

Histological examination of the spheres incubated in IGF or control medium revealed the presence of occasional epithelial-lined tubular or cystic structures. In contrast, samples treated with TGF-β, activin-A, or RA did not contain any such structures, individual cells were smaller, and there was generally less overall extracellular matrix produced (FIG. 2A). Spheres in the latter groups appeared deteriorated, with the least cellular viability in activin-A treated samples. Although matrigel supported formation of some tubular or cystic structures with open lumens when treated with IGF or control medium, cellular degeneration, deformation of shape, and variation in spheres sizes all suggested that matrigel alone was insufficient for supporting hES cell growth and 3D organization.

Scaffolds Provide Mechanical Support to Withstand hES Cell Contraction

Biodegradable scaffolds were used to create a 3D supportive environment for directing differentiation and organization of hES cells into tissue-like structures. Scaffolds were fabricated from a blend of 50% poly(lactic-co-glycolic acid) (PLGA) and 50% poly(L-lactic acid) (PLLA). The PLGA was selected to degrade quickly (approximately 3 weeks) to facilitate cellular ingrowth, while the PLLA was chosen to provide mechanical stiffness to resist the contractile forces of the cells. A pore size of 250-500 μm was chosen to facilitate the seeding and ingrowth of the cells.

To determine whether the scaffold would withstand the mechanical force exerted by the cells, we carried out compressive and tensile tests. The compressive tests were performed on the PLLA/PLGA scaffolds alone and with Growth Factor-Reduced Matrigel, and the results are summarized in FIG. 2B-C. These data were then compared to published values for matrigel alone (FIG. 2D)¹⁶. The scaffold showed tensile properties consistent with previously reported values for high molecular weight PLLA scaffolds (FIG. 2B,D)¹⁷. In compression, the polymer scaffold had a compressive modulus of approximately 65 kPa. The addition of matrigel did not alter the compressive modulus, as determined by statistical analysis using ANOVA (FIG. 2C,D) The summary table (FIG. 2D) demonstrates that the scaffold and the matrigel/scaffold exhibit a compressive modulus three orders of magnitude greater than that of matrigel alone. This difference influences the performance of the scaffold with cells. At an estimated compressive cell stress of 110 Pa, the scaffold will contract by 0.2 percent, meaning that it will essentially resist contraction.

Scaffolds Support hES Cell Attachment Growth, Differentiation, and 3D Orqanization

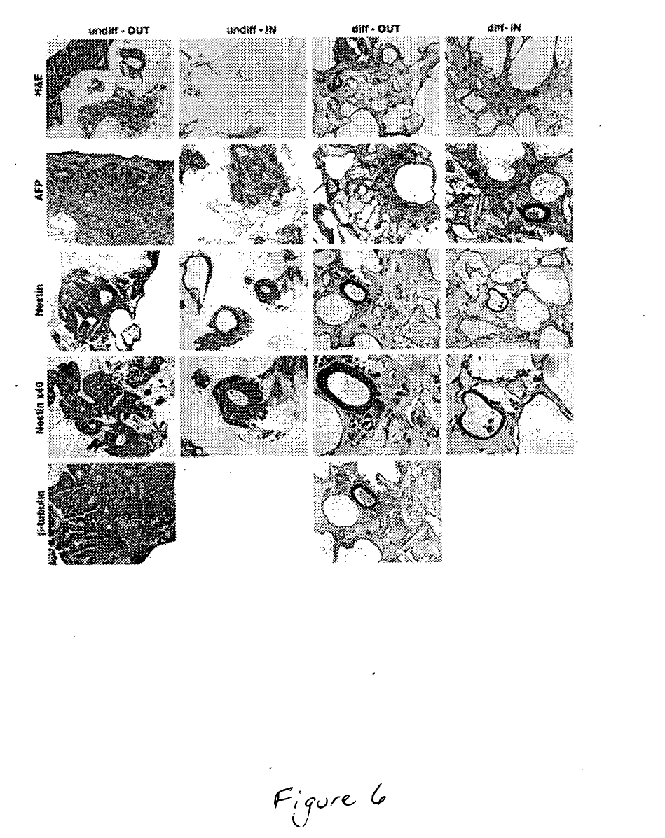

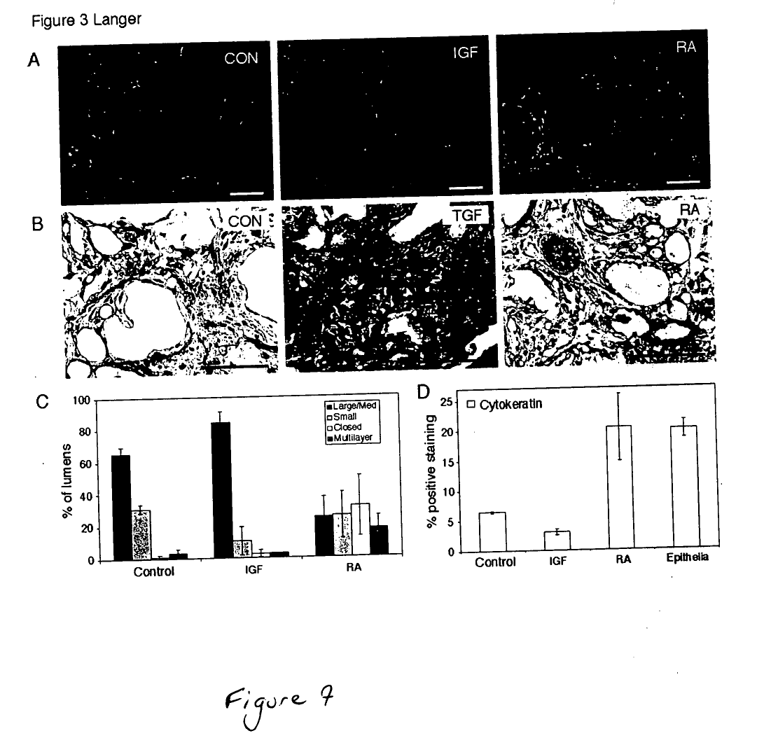

To determine whether the scaffold had an effect on hES cell differentiation and 3D organization, we compared 2-week incubations of differentiating hES cells cultured on fibronectin-coated dishes versus fibronectin-coated scaffolds, as well as differentiation in matrigel alone versus matrigel with scaffold. The two-dimensional fibronectin-coated dish supported some cell differentiation (FIG. 3) but could not support 3D structure formation. Matrigel alone could form a 3D environment, but it failed to support hES cell growth and 3D organization (FIG. 2). One possibility is that the differences obtained between matrigel alone and scaffolds with matrigel could partially be caused by the scaffold's mechanical stiffness, which is necessary to resist the force of cell contraction.

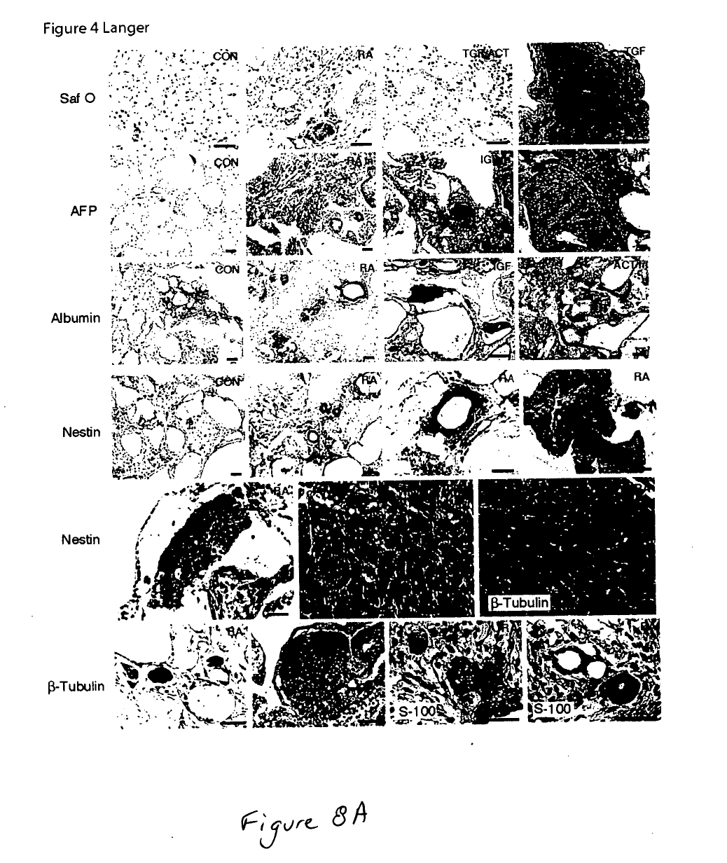

When comparing differentiation and organization of scaffold grown constructs versus EBs, we found higher expression of differentiation-associated proteins such as cytokeratin, AFP, and nestin on the scaffolds, which correlated with more organization into defined epithelial tubular structures and neural tube-like rosettes (FIG. 4). Regarding extracellular matrix production, no safranin-O staining was observed in EBs conditioned with TGF-β. The EB population was very heterogeneous in structure and protein expression levels. Consequently, polymer scaffolds appeared to be more suitable than EBs in promoting cell differentiation and homogeneity.

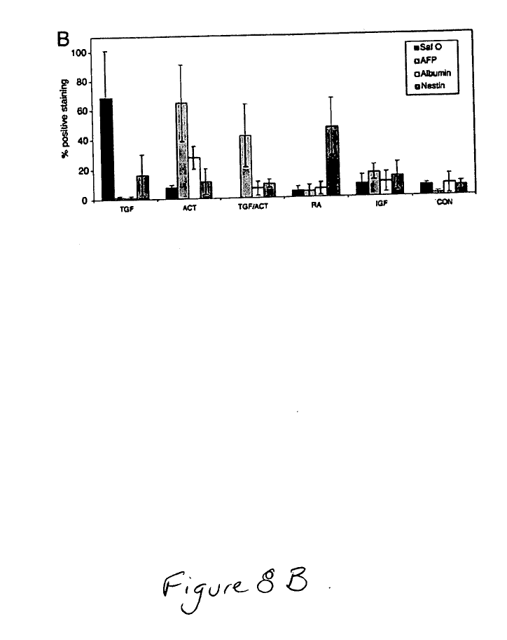

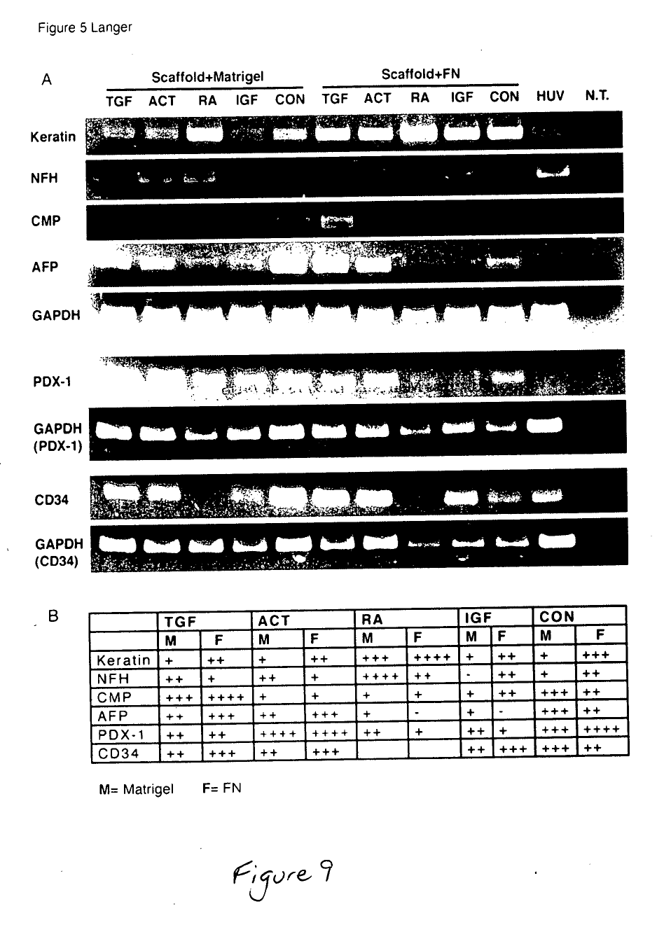

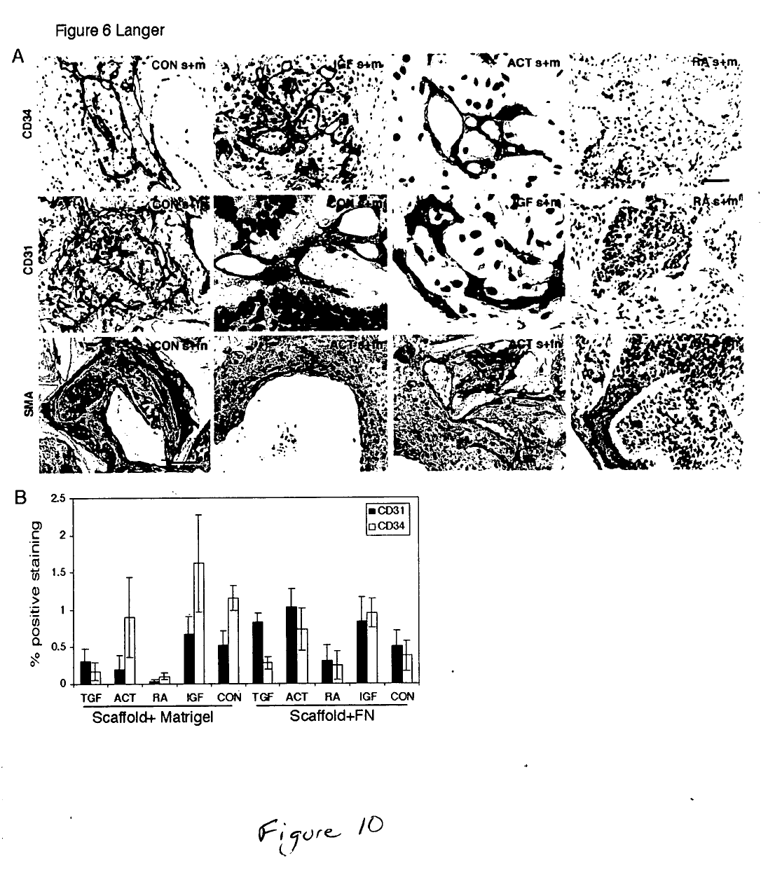

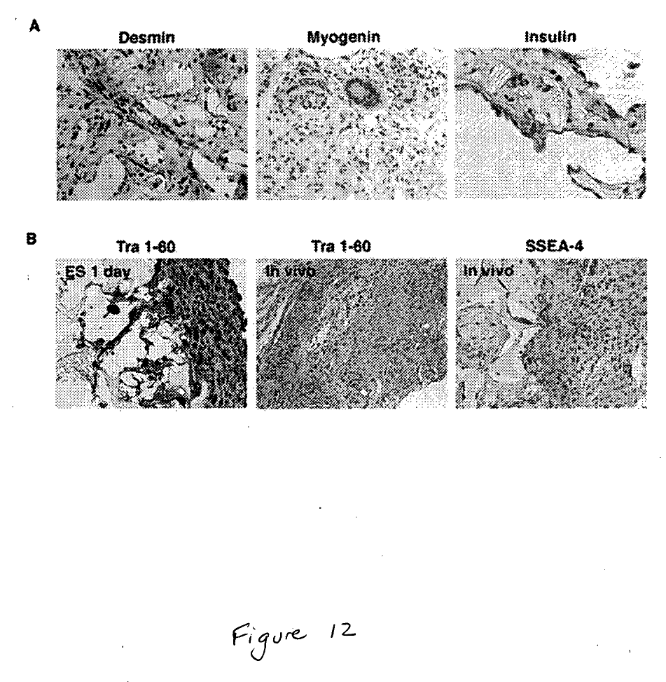

Both matrigel (FIG. 5E,G) and fibronectin (FIG. 5F,H) promoted anchorage of the differentiating hES (EB day 8) cells onto the scaffolds, growth and cell viability. The cells attached throughout the inner and outer surfaces of the scaffold, filling the pores, as shown by scanning electron microscopy (FIG. 5A-D) and routine histology of tissue sections taken at different depths (FIG. 5E-H). After the two-week period, constructs incubated with BrdUrd showed high levels of proliferation and viability throughout the scaffold (FIG. 5I-K). Differentiating hES cells were used instead of undifferentiated hES cells based on observations that scaffolds seeded with undifferentiated hES cells exhibited clear perforation of the outer surfaces and less uniform growth and survival in the center of the scaffolds when compared with differentiating hES cells (EB day 8) (FIG. 6, see also FIG. 12A).

Following the incubation period, samples organized into 3D patterns that resembled tissue structures. To assess these structures, we analyzed formation and organization of epithelial and mesenchymal structures and extracellular matrix (FIG. 7). Addition of IGF resulted in formation of relatively large tubulocystic structures (84%±6>40 μm, 10%±3>200 μm) lined by cytokeratin-positive cuboidal-to-columnar epithelial cells when compared to the control medium with no growth factor supplementation (65%±4>40 μm) (P<0.01). In contrast, RA induced formation of structures with lumens that were smaller than that of control samples (25%±12>40 μm) (P<0.01) and often produced circular multilayered or closed bodies (FIG. 7A,C). RA treatment resulted in a ˜4-fold increase in the total percentage of cytokeratin-positive areas within the tissue (P<0.01), approaching a level found in an adult epithelial tissue tested (FIG. 7D). The cellular structures secreted extracellular matrix components into their surroundings, as indicated by trichrome staining for collagen (FIG. 7B). Collagen formation in the matrix and the organization of the matrix between the cells were dramatically affected by addition of growth factors (FIG. 7B). Newly formed poorly organized collagen in control medium is lightly fibrillar and weak staining. Addition of TGFβ to the medium induced mature collagen formation with thick densely staining bands, while RA inhibited collagen formation. Regardless of conditions, tubulocystic structures and extracellular matrix production in scaffold-supported culture systems were larger and better differentiated than structures in equivalently-treated samples with matrigel alone.

Engineering 3D Mesodermal, Ectodermal and Endodermal Tissue Structures Using Biodegradable Polymer Scaffolds

We further investigated the role of chemical cues coupled with physical cues to promote differentiation into specific mesodermal, ectodermal, and endodermal-derived tissue structures. Based on studies on the differentiation of mouse and human ES cells in EB models and monolayers⁶⁻⁸, we chose growth factors known to induce differentiation into specific germ layer(s).

To induce mesodermal tissue formation, we incubated the cells for two weeks with TGF-β, activin-A or a combination of TGF-β and activin-A. Addition of TGF-β to the medium induced formation of cartilaginous tissue throughout the whole construct, as indicated by high levels of Safranin-O staining for the glycosaminoglycans (GAG), characteristic of cartilage extracellular matrix¹⁸ (FIG. 8). In contrast, addition of other growth factors such as activin-A (even when added together with TGF-β), IGF, and RA did not induce formation of Safranin O-positive matrix (FIG. 8). RT-PCR analysis of RNA extracted from the different constructs indicated higher levels of cartilage matrix protein (CMP) expression in samples treated with TGF-β, compared to the other samples (FIG. 9A). To our knowledge, these results demonstrate for the first time the formation of 3D cartilage-like tissue using differentiating hES cells.

Addition of activin-A or IGF both induced the formation of structures with biochemical features of developing liver. In comparison to the control, activin-A induced high levels of alpha feto protein (AFP) and albumin throughout the sample. IGF induced high levels of AFP and albumin in more defined areas within the constructs (FIG. 8), while no staining was observed with the addition of RA. These results suggest that in scaffold-supported hES 3D constructs, activin-A and IGF can induce endodermal differentiation and formation of tissue with a biochemical profile consistent with developing liver. Gene expression analysis indicated higher levels of the pancreatic gene PDX-1 in tissue-constructs that were treated with activin-A, than with other growth factors (FIG. 9B), which further supported the role of activin-A in inducing differentiation of hES cells into endodermal-derived tissues on polymer scaffolds.

For ectodermal structures, we added RA to the construct medium^{7,8,19}. In comparison to other growth factors, RA supplementation resulted in preferential development of epithelial-lined solid and ductular structures (FIG. 7). Moreover, staining with neural markers indicated that the cells organized into single or large multilayered neural tube-like rosette structures that were positive for nestin and β_{III}-tubulin. Large areas without features of rosettes also stained positive for nestin and β_{III}-tubulin (FIG. 8). Cells stained for S-100, a marker for glial and other neuroectodermal cells, surrounded some of the tubes, suggesting a supportive or migratory phenotype. Gene expression analysis of samples treated with RA indicated high levels of keratin and neurofilament RNA and very low expression of mesodermal and endodermal genes, in contrast to other samples (FIG. 9). These results show that RA induces ectodermal differentiation of hES grown on polymer scaffolds, with a predilection for development of higher-order structures morphologically and biochemically consistent with nervous tissue.

Analysis of the tissue structures formed in matrigel alone showed that chemical factors did not induce differentiation as seen on scaffolds. Instead of forming ductular and rosette-like structures in the presence of RA, the cells on matrigel organized into small clusters, which had very low expression (if any) of nestin. No AFP expression was observed in the activin-A treated matrigel samples. In IGF and control samples, some AFP staining could be observed. No Safanin-O staining of cartilage-derived GAG was observed in the TGF-β treated samples (FIG. 2). These results show that the scaffold is influential in promoting the formation of three-dimensional cartilage, liver and neural-like tissues in vitro.

Vascularization of Three-Dimensional Tissue Constructs in vitro.

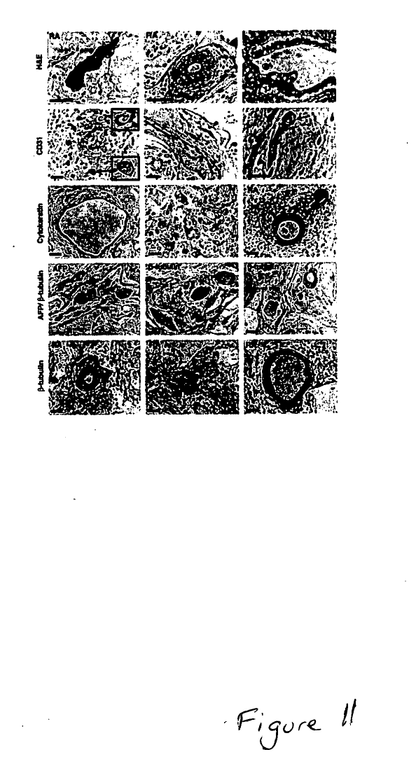

Since blood vessels facilitate the formation of complex tissue structures²⁰⁻²², we analyzed whether hES cells were able to differentiate and organize into blood vessels within the tissue structures formed on the scaffold. Staining with antibodies against CD34 and CD31 indicated that following the two-week incubation period with the scaffolds, the cells differentiated into endothelial cells and, moreover, organized into vessel-like structures throughout the tissue. 3D culture of the cells promoted formation of massive 3D vascular networks that closely interacted with the surrounding tissue (FIG. 10). Comparison of vascularization in the scaffolds in the presence and absence of matrigel indicated that matrigel was not required, as samples seeded on fibronectin-coated scaffolds (without matrigel) resulted in higher levels of endothelial differentiation and vascularization (FIG. 10). Interestingly, samples that were treated with RA neither formed vessels (indicated by immunostaining with CD34 and CD31) nor expressed CD34 or CD31 genes as shown by RNA analysis (FIG. 9, 10). Elongated smooth muscle-like cells were also detected. These were organized around some lumens within the tissue, but not in samples treated with RA (FIG. 10). These results indicate that differentiating hES cells grown on polymer scaffolds can differentiate and form vascularized complex tissue structures. Furthermore, this in vitro vascularization process, provided with the scaffold's physical guidance, can be controlled by addition of growth factors to the culture medium.

Evaluation of Three-Dimensional Tissue Constructs After Two Weeks in vivo

To analyze the therapeutic potential of hES-derived polymer scaffold constructs, we surgically implanted 2-week-old constructs into s.c. tissue of SCID mice. At the time of implant retrieval (14 days after implantation), cells within constructs were viable and no signs of infection were detected. Implants were incompletely encapsulated by loose fibrogranulomatous connective tissue and permeated with host blood vessels. Immunohistochemical staining, using human-specific CD31 antibodies, demonstrated the presence of both immunoreactive (construct, FIG. 11, arrows) and nonimmunoreactive (host, FIG. 11, arrowheads) vessels throughout the constructs. Moreover, construct-derived vessels contained intraluminal red blood cells, suggesting construct-host vascular anastamosis. Immunostaining with cytokeratin, β_{III}-tubulin, and AFP antibodies indicated that the implanted constructs continued to express these human proteins in defined structures within the scaffold area (FIG. 11). In certain instances there appeared to be continued differentiation and organization of constructs after implantation (FIG. 11), which was affected by the specific cytokine treatment before implantation.

After continued construct maturation in vivo, RA-conditioned constructs exhibited larger and better organized neural structures than those seen in vitro (or with control medium in vitro or in vivo) including ductular structures lined by tall columnar epithelium invested with long cilia resembling ependymal cells and rosettes with abundant melanin granules (brown/black in H&E section; confirmed by potassium permanganate staining, data not shown). β_{III}-tubulin antibodies stained neuroectodermal structures within the implant as well as murine peripheral nerve fibers in surrounding connective tissue (FIG. 11, asterisk). Staining with SSEA-4 and Tra 1-60 antibodies indicated that none of the cells remained undifferentiated (FIG. 12B).

Discussion

Both the physical environment and appropriate growth factor supplementation are important in the formation of human tissue-like 3D structures. We have demonstrated formation of tissues with morphologic and biochemical features consistent with developing human cartilage, liver, nerve and blood vessels in vitro, using hES cells grown on polymer scaffolds. We found that the scaffold promoted the formation of differentiated tissues. Using contractile forces of fibroblasts to model cellular behavior on a scaffold, cellular stress was estimated to be 110 Pa. Under this stress, matrigel will contract by 700 percent while the scaffold will contract by only 0.2 percent, meaning the scaffold essentially would not contract. Depending on the cell type, however, cells may display different contractile forces. In addition, the chemical environment also plays a role in mechanical behavior of cells. It has been shown that growth factors affect the mechanical behavior of cells, including stem cells²³⁻²⁶. This may explain why matrigel contracted less under some growth factor conditions (IGF, or control medium), but totally collapsed under others (activin-A, RA) (FIG. 2). When cells were grown on scaffolds with the same growth factor supplementation, further differentiation was induced into various specific cell types (such as endothelial, neuronal, hepatocytes, etc), with organization into 3D tissue structures (such as blood-vessel networks, neural tube-like structures etc.)(FIG. 8-10). These findings suggest that both chemcial and physical cues (e.g., mechanical support provided by the scaffolds) influence differentiation of ES cells to complex tissues.

The effects of the growth factors may result from direct differentiation or from cell selection by either promoting or inhibiting proliferation or by inducing apoptosis of specific cell types. For example, when cells were seeded on scaffolds, RA treatment induced specific differentiation into epithelial and neural-like structures and inhibited mesodermal and endodermal differentiation (FIG. 8-10). The addition of activin-A to hES cells grown on the scaffolds induced significant endodermal differentiation, as shown by immunostaining with AFP and albumin, two major proteins characteristics of hepatic differentiation^{27,28}, and by expression of the pancreatic gene PDX-1²⁹ (FIG. 8, 9). Activin-A is known as mainly a mesodermal factor^{6,30}, and in the hES monolayer cell system has been shown to induce mainly mesoderm (mainly muscle) differentiation with no expression of any tested endodermal (including AFP and albumin) or ectodermal genes⁷. However there are reports showing that activin-A can induce endodermal differentiation³¹⁻³³. It is possible that the timing of application (EB day 8 versus day 5) or the three-dimensionality plays a role in the effect of activin-A on hES cell differentiation. Another explanation for the differences in activin-A effect between the two systems could be due to the fact that the 3D structures supported tissue vascularization (in conditions that allowed mesodermal differentiation). It was shown recently that endothelial cells and nascent vessels (even prior to blood vessel function) provide inductive signals that are important for liver and pancreatic development^{34,35}. Therefore, formation of a blood vessel network on the scaffolds could support an inductive effect of activin-A toward endodermal differentiation.

These results indicate that complex structures with features of various committed embryonic tissues can be generated, in vitro, by using early differentiating hES cells and further inducing their differentiation in a supportive 3D environment such as PLLA/PLGA polymer scaffolds. The in vivo results show that scaffold-supported hES constructs remain viable for at least 2 weeks, that constructs may recruit and anastamose with the host vascular system, and that the differentiation pattern induced in vitro remains intact or continues to progress in vivo. Growth of human tissues in vitro holds promise for addressing organ shortages and infectious disease risks, which present serious challenges in transplantation medicine. In addition to potential clinical applications, in vitro tissue formation may provide an important tool for studying early human development and organogenesis.

### REFERENCES

1. Dushnik-Levinson, M. & Benvenisty, N. Embryogenesis in vitro: study of differentiation of embryonic stem cells. Biol Neonate 67, 77-83 (1995).
2. Thomson, J. A. et al. Embryonic stem cell lines derived from human blastocysts. Science 282, 1145-1147 (1998).
3. Wobus, A. M. Potential of embryonic stem cells. Mol Aspects Med 22, 149-164 (2001).
4. Stocum, D. L. Stem cells in regenerative biology and medicine. Wound Repair Regen 9, 429-442 (2001).
5. Itskovitz-Eldor, J. et al. Differentiation of human embryonic stem cells into embryoid bodies compromising the three embryonic germ layers. Mol Med 6, 88-95 (2000).
6. Johansson, B. M. & Wiles, M. V. Evidence for involvement of activin A and bone morphogenetic protein 4 in mammalian mesoderm and hematopoietic development. Mol Cell Biol 15, 141-151 (1995).
7. Schuldiner, M., Yanuka, O., Itskovitz-Eldor, J., Melton, D. A. & Benvenisty, N. From the cover: effects of eight growth factors on the differentiation of cells derived from human embryonic stem cells. Proc Natl Acad Sci USA 97, 11307-11312 (2000).
8. Guan, K., Chang, H., Rolletschek, A. & Wobus, A. M. Embryonic stem cell-derived neurogenesis. Retinoic acid induction and lineage selection of neuronal cells. Cell Tissue Res 305, 171-176 (2001).
9. Kaufman, D. S., Hanson, E. T., Lewis, R. L., Auerbach, R. & Thomson, J. A. Hematopoietic colony-forming cells derived from human embryonic stem cells. Proc Natl Acad Sci USA 98, 10716-10721 (2001).
10. Ito, Y. Surface micropatterning to regulate cell functions. Biomaterials 20, 2333-2342 (1999).
11. Ballermann, B. J., Dardik, A., Eng, E. & Liu, A. Shear stress and the endothelium. Kidney Int Suppl 67, S100-108 (1998).
12. Carter, D. R., Beaupre, G. S., Giori, N. J. & Helms, J. A. Mechanobiology of skeletal regeneration. Clin Orthop, S41-55 (1998).
13. Ingber, D. E. & Folkman, J. Mechanochemical switching between growth and differentiation during fibroblast growth factor-stimulated angiogenesis in vitro: role of extracellular matrix. J Cell Biol 109, 317-330 (1989).
14. Darland, D. C. & D'Amore, P. A. TGF beta is required for the formation of capillary-like structures in three-dimensional cocultures of IOT1/2 and endothelial cells. Angiogenesis 4, 11-20(2001).
15. Levenberg, S., Golub, J. S., Amit, M., Itskovitz-Eldor, J. & Langer, R. Endothelial cells derived from human embryonic stem cells. Proc Natl Acad Sci USA 99, 4391-4396 (2002).
16. Semler, E. J., Ranucci, C. S. & Moghe, P. V. Mechanochemical manipulation of hepatocyte aggregation can selectively induce or repress liver-specific function. Biotechnol Bioeng 69, 359-369 (2000).
17. Pegoretti, A., Fambri, L. & Migliaresi, C. In vitro degradation of poly(L-lactic acid) fibers produced by melt spinning. J Appl Polym Sci 64, 213-223 (1996).
18. Naumann, A. et al. Immunochemical and mechanical characterization of cartilage subtypes in rabbit. J Histochem Cytochem 50, 1049-1058 (2002).
19. Dinsmore, J. et al. Embryonic stem cells differentiated in vitro as a novel source of cells for transplantation. Cell Transplant 5, 131-143 (1996).
20. Risau, W. & Flamme, I. Vasculogenesis. Annu Rev Cell Dev Biol 11, 73-91 (1995).
21. Folkman, J. & D'Amore, P. A. Blood vessel formation: what is its molecular basis? Cell 87, 1153-1155 (1996).
22. Nomi, M., Atala, A., Coppi, P. D. & Soker, S. Principals of neovascularization for tissue engineering. Mol Aspects Med 23, 463 (2002).
23. Allen, F. D. et al. Epidermal growth factor induces acute matrix contraction and subsequent calpain-modulated relaxation. Wound Repair Regen 10, 67-76 (2002).
24. Kinner, B., Zaleskas, J. M. & Spector, M. Regulation of smooth muscle actin expression and contraction in adult human mesenchymal stem cells. Exp Cell Res 278, 72-83 (2002).
25. Semler, E. J. & Moghe, P. V. Engineering hepatocyte functional fate through growth factor dynamics: the role of cell morphologic priming. Biotechnol Bioeng 75, 510-520 (2001).
26. Zaleskas, J. M. et al. Growth factor regulation of smooth muscle actin expression and contraction of human articular chondrocytes and meniscal cells in a collagen-GAG matrix. Exp Cell Res 270, 21-31 (2001).
27. Abe, K. et al. Endoderm-specific gene expression in embryonic stem cells differentiated to embryoid bodies. Exp Cell Res 229, 27-34 (1996).
28. Hamazaki, T. et al. Hepatic maturation in differentiating embryonic stem cells in vitro. FEBS Lett 497, 15-19 (2001).
29. Offield, M. F. et al. PDX-1 is required for pancreatic outgrowth and differentiation of the rostral duodenum. Development 122, 983-995 (1996).
30. Smith, J. C., Price, B. M., Van Nimmen, K. & Huylebroeck, D. Identification of a potent Xenopus mesoderm-inducing factor as a homologue of activin A. Nature 345, 729-731 (1990).
31. Manova, K., Paynton, B. V. & Bachvarova, R. F. Expression of activins and TGF beta 1 and beta 2 RNAs in early postimplantation mouse embryos and uterine decidua. Mech Dev 36, 141-152 (1992).
32. Ninomiya, H., Takahashi, S., Tanegashima, K., Yokota, C. & Asashima, M. Endoderm differentiation and inductive effect of activin-treated ectoderm in Xenopus. Dev Growth Differ 41, 391-400 (1999).
33. Tiedemann, H., Asashima, M., Grunz, H. & Knochel, W. Pluripotent cells (stem cells) and their determination and differentiation in early vertebrate embryogenesis. Dev Growth Differ 43, 469-502 (2001).
34. Lammert, E., Cleaver, 0. & Melton, D. Induction of pancreatic differentiation by signals from blood vessels. Science 294, 564-567 (2001).
35. Matsumoto, K., Yoshitomi, H., Rossant, J. & Zaret, K. S. Liver organogenesis promoted by endothelial cells prior to vascular function. Science 294, 559-563 (2001).
36. Donovan, P. J. & Gearhart, J. The end of the beginning for pluripotent stem cells. Nature 414, 92-97 (2001).
37. Odorico, J. S., Kaufman, D. S. & Thomson, J. A. Multilineage differentiation from human embryonic stem cell lines. Stem Cells 19, 193-204 (2001).
38. Freyman, T. M., Yannas, I. V., Yokoo, R. & Gibson, L. J. Fibroblast contractile force is independent of the stiffness which resists the contraction. Exp Cell Res 272, 153-162 (2002).

Other embodiments of the invention will be apparent to those skilled in the art from a consideration of the specification or practice of the invention disclosed herein. It is intended that the specification and examples be considered as exemplary only, with the true scope and spirit of the invention being indicated by the following claims.

1. A tissue engineering construct, comprising:
embryonic stem cells;
a three-dimensional cell support matrix, wherein the matrix is resistant to contractile forces exerted by the stem cells; and
at least one growth factor selected to promote differentiation of the stem cells along a predetermined cell lineage or into a specific cell type.

2. The tissue engineering construct of claim 1, wherein the stem cells are mammalian embryonic stem cells.

3. The tissue engineering construct of claim 2, wherein the cells are human embryonic stem cells.

4. The tissue engineering construct of claim 1, wherein the cell support matrix comprises a poly(lactic acid)-poly(lactic acid-co-glycolic acid) mixture.

5. The tissue engineering construct of claim 4, wherein the cell support matrix comprises a 50/50 mixture of poly(L-lactic acid) and poly(lactic acid-co-glycolic acid).

6. The tissue engineering construct of claim 1, wherein a cross-sectional area of the matrix is not reduced by more than 50% under a contractile force exerted by the embryonic stem cells.

7. The tissue engineering construct of claim 6, wherein a cross-sectional area of the matrix is not reduced by more than 40% under a contractile force exerted by the embryonic stem cells.

8. The tissue engineering construct of claim 7, wherein a cross-sectional area of the matrix is not reduced by more than 30% under a contractile force exerted by the embryonic stem cells.

9. The tissue engineering construct of claim 8, wherein a cross-sectional area of the matrix is not reduced by more than 20% under a contractile force exerted by the embryonic stem cells.

10. The tissue engineering construct of claim 9, wherein a cross-sectional area of the matrix is not reduced by more than 10% under a contractile force exerted by the embryonic stem cells.

11. The tissue engineering construct of claim 10, wherein a cross-sectional area of the matrix is not reduced by more than 1% under a contractile force exerted by the embryonic stem cells.

12. The tissue engineering construct of claim 1, wherein the cell support matrix further comprises a coating including an agent that promotes cell adhesion.

13. The tissue engineering construct of claim 12, wherein the agent that promotes cell adhesion is selected from fibronectin, integrins, and oligonucleotides that promote cell adhesion.

14. The tissue engineering construct of claim 1, wherein the cell support matrix is biodegradable or non-biodegradable.

15. The tissue engineering construct of claim 14, wherein the cell support matrix is selected from PLA, PGA, PLGA, poly(anhydrides), poly(hydroxy acids), poly(ortho esters), poly(propylfumerates), poly(caprolactones), polyamides, polyamino acids, polyacetals, biodegradable polycyanoacrylates, biodegradable polyurethanes, polysaccharides, polypyrrole, polyanilines, polythiophene, polystyrene, polyesters, non-biodegradable polyurethanes, polyureas, poly(ethylene vinyl acetate), polypropylene, polymethacrylate, polyethylene, polycarbonates, poly(ethylene oxide), co-polymers of any of the above, adducts of any of the above, and mixtures of any of the above polymers, co-polymers, and adducts with one another.

16. The tissue engineering construct of claim 1, further comprising one or more biomolecules, small molecules, or bioactive agents disposed within the cell support matrix.

17. The tissue engineering construct of claim 1, further comprising a gel that coats internal and external surfaces of the cell support matrix.

18. The tissue engineering construct of claim 17, wherein the gel is selected from collagen gel, alginate, agar, and Growth Factor Reduced MATRIGEL™.

19. The tissue engineering construct of claim 18, wherein the gel further comprises one or more of laminin, fibrin, fibronectin, proteoglycans, glycoproteins, glycosaminoglycans, chemotactic agents, or growth factors.

20. The tissue engineering construct of claim 1, wherein the growth factor is selected from cytokines, eicosanoids, and differentiation factors.

21. The tissue engineering construct of claim 20, wherein the growth factor is selected from activin-A (ACT), retinoic acid (RA), epidermal growth factor, bone morphogenetic protein, TGF-β, hepatocyte growth factor, platelet-derived growth factor, TGF-α, IGF-I and II, hematopoietic growth factors, heparin binding growth factor, peptide growth factors, erythropoietin, interleukins, tumor necrosis factors, interferons, colony stimulating factors, fibroblast growth factors, nerve growth factor (NGF) and muscle morphogenic factor (MMF).

22. The tissue engineering construct of claim 1, wherein the cell support matrix has a shape selected from particles, tube, sponge, sphere, strand, coiled strand, capillary network, film, fiber, mesh, and sheet.

23. A method of producing a tissue engineering construct, comprising:
providing a population of embryonic stem cells;
seeding the embryonic stem cells on a cell support matrix; and
exposing the embryonic stem cells to at least one agent selected to promote differentiation of the stem cells along a predetermined cell lineage or into a specific cell type,
wherein the step of exposing may be performed before or after the step of seeding, or both.

24. The method of claim 23, wherein the embryonic stem cells are mammalian embryonic stem cells.

25. The method of claim 24, wherein the embryonic stem cells are human embryonic stem cells.

26. The method of claim 23, wherein the cell support matrix is three dimensional.

27. The method of claim 23, wherein a cross-sectional area of the matrix is not reduced by more than 50% under a contractile force exerted by the embryonic stem cells.

28. The method of claim 27, wherein a cross-sectional area of the matrix is not reduced by more than 40% under a contractile force exerted by the embryonic stem cells.

29. The method of claim 28, wherein a cross-sectional area of the matrix is not reduced by more than 30% under a contractile force exerted by the embryonic stem cells.

30. The method of claim 29, wherein a cross-sectional area of the matrix is not reduced by more than 20% under a contractile force exerted by the embryonic stem cells.

31. The method of claim 30, wherein a cross-sectional area of the matrix is not reduced by more than 10% under a contractile force exerted by the embryonic stem cells.

32. The method of claim 31, wherein a cross-sectional area of the matrix is not reduced by more than 1% under a contractile force exerted by the embryonic stem cells.

33. The method of claim 23, wherein the cell support matrix comprises a poly(lactic acid)-poly(lactic acid-co-glycolic acid) mixture.

34. The method of claim 33, wherein the cell support matrix comprises a 50/50 mixture of poly(L-lactic acid) and poly(lactic acid-co-glycolic acid).

35. The method of claim 23, further comprising coating the cell support matrix with an agent that promotes cell adhesion.

36. The method of claim 35, wherein the agent that promotes cell adhesion is selected from fibronectin, integrins, and oligonucleotides that promote cell adhesion.

37. The method of claim 23, wherein the cell support matrix is biodegradable or non-biodegradable.

38. The method of claim 23, wherein the cell support matrix is selected from PLA, PGA, PLGA poly(anhydrides), poly(hydroxy acids), poly(ortho esters), poly(propylfumerates), poly(caprolactones), polyamides, polyamino acids, polyacetals, biodegradable polycyanoacrylates, biodegradable polyurethanes, polysaccharides, polypyrrole, polyanilines, polythiophene, polystyrene, polyesters, non-biodegradable polyurethanes, polyureas, poly(ethylene vinyl acetate), polypropylene, polymethacrylate, polyethylene, polycarbonates, poly(ethylene oxide), co-polymers of any of the above, adducts of any of the above, and mixtures of any of the above polymers, co-polymers, and adducts with one another.

39. The method of claim 23, further comprising adding one or more biomolecules, small molecules, and bioactive agents to the cell support matrix.

40. The method of claim 23, further comprising disposing the embryonic stem cells within a gel, wherein seeding the embryonic stem cells on the cell support matrix includes disposing the gel on internal and external surfaces of the cell support matrix.

41. The method of claim 40, wherein the gel is selected from collagen gel, alginate, agar, and Growth Factor Reduced MATRIGEL™.

42. The method of claim 41, wherein the gel further comprises one or more of laminin, fibrin, fibronectin, proteoglycans, glycoproteins, glycosaminoglycans, chemotactic agents, and growth factors.

43. The method of claim 23, wherein culturing is conducted in a serum-free medium.

44. The method of claim 23, wherein the agent is selected from a growth factor, a mechanical force, an electric voltage, a bioactive agent, a biomolecule, and a small molecule.

45. The method of claim 44, wherein the growth factor is selected from cytokines, eicosanoids, and differentiation factors.

46. The method of claim 45, wherein the growth factor is selected from activin-A (ACT), retinoic acid (RA), epidermal growth factor, bone morphogenetic protein, TGF-β, hepatocyte growth factor, platelet-derived growth factor, TGF-α, IGF-I and II, hematopoietic growth factors, heparin binding growth factor, peptide growth factors, erythropoietin, interleukins, tumor necrosis factors, interferons, colony stimulating factors, fibroblast growth factors, nerve growth factor (NGF) and muscle morphogenic factor (MMF).

47. The method of claim 44, wherein the mechanical force is selected from hoop stress, shear stress, hydrostatic stress, compressive stress, tensile stress, and combinations of the above.

48. The method of claim 23, wherein the cell support matrix has a shape selected from particles, tube, sponge, sphere, strand, coiled strand, capillary network, film, fiber, mesh, and sheet.

49. The method of claim 23, wherein providing includes culturing embryonic stem cells in the presence of a growth factor.

50. The method of claim 49, wherein culturing is conducted in a serum-free medium.

51. A tissue engineering construct, comprising:
embryonic stem cells;
a three-dimensional cell support matrix comprising a 50/50 mixture of poly (L-lactic acid) and poly (lactic-co-glycolic acid); and
TGF-β.

52. A tissue engineering construct, comprising:
embryonic stem cells;
a three-dimensional cell support matrix comprising a 50/50 mixture of poly (L-lactic acid) and poly (lactic-co-glycolic acid); and
a member of activin A, IGF, and any combination of the above.

53. A tissue engineering construct, comprising:
embryonic stem cells;
a three-dimensional cell support matrix comprising a 50/50 mixture of poly (L-lactic acid) and poly (lactic-co-glycolic acid); and
retinoic acid.

54. The tissue engineering construct of claim 51, 52, or 53, wherein the cell support matrix further comprises one or more of fibronectin or growth factor-reduced MATRIGEL.

55. A method of promoting tissue development, comprising:
providing a population of embryonic stem cells;
seeding the embryonic stem cells on a cell support matrix comprising a 50/50 mixture of poly(L-lactic acid) and poly(lactic-co-glycolic acid); and
exposing the embryonic stem cells to TGF-β,
wherein the cells produce cartilaginous tissue.

56. A method of promoting tissue development, comprising;
providing a population of embryonic stem cells;
seeding the embryonic stem cells on a cell support matrix comprising a 50/50 mixture of poly(L-lactic acid) and poly(lactic-co-glycolic-acid); and
exposing the embryonic stem cells to one or more of activin A and IGF,
wherein the cells produce alpha feto protein and albumin.

57. A method of promoting tissue development, comprising:
providing a population of embryonic stem cells;
seeding the embryonic stem cells on a cell support matrix comprising a 50/50 mixture of poly (L-lactic acid) and poly (lactic-co-glycolic acid); and
exposing the embryonic stem cells to retinoic acid,
wherein the cells develop neuronal tissue structures.

58. The method of claims 55, 56, or 57 wherein the cell support matrix further comprises one or more of fibronectin or Growth Factor-Reduced MATRIGE™.

59. The method of claims 55, 56, or 57, wherein exposing comprises culturing the seeded cell support matrix in vitro for two weeks and the method further comprises implanting the seeded cell support matrix in an animal.

60. A method of promoting tissue development, comprising:
providing a population of embryonic stem cells;
seeding the embryonic stem cells on a cell support matrix;
culturing the seeded cell support matrix in the presence of a growth factor for a predetermined amount of time; and
implanting the cultured cell support matrix in an animal.

61. The method of claim 60, wherein the cell support matrix is selected from PLA, PGA, PLGA poly(anhydrides), poly(hydroxy acids), poly(ortho esters), poly(propylfumerates), poly(caprolactones), polyamides, polyamino acids, polyacetals, biodegradable polycyanoacrylates, biodegradable polyurethanes, polysaccharides, polypyrrole, polyanilines, polythiophene, polystyrene, polyesters, non-biodegradable polyurethanes, polyureas, poly(ethylene vinyl acetate), polypropylene, polymethacrylate, polyethylene, polycarbonates, poly(ethylene oxide), co-polymers of any of the above, adducts of any of the above, and mixtures of any of the above polymers, co-polymers, and adducts with one another.

62. The method of claim 60, wherein the three-dimensional cell support matrix comprises a 50/50 mixture of poly (L-lactic acid) and poly (lactic-co-glycolic acid).

63. The method of claim 60, further comprising coating the cell support matrix with an agent that promotes cell adhesion.

64. The method of claim 63, wherein the agent that promotes cell adhesion is selected from fibronectin, integrins, and oligonucleotides that promote cell adhesion.

65. The method of claim 60, further comprising disposing the embryonic stem cells within a gel, wherein seeding the embryonic stem cells on the cell support matrix includes disposing the gel on internal and external surfaces of the cell support matrix.

66. The method of claim 65, wherein the gel is selected from collagen gel, alginate, agar, and Growth Factor Reduced MATRIGEL™.

67. The method of claim 65, wherein the gel further comprises one or more of laminin, fibrin, fibronectin, proteoglycans, glycoproteins, glycosaminoglycans, chemotactic agents, and growth factors.

68. The method of claim 60, wherein the growth factor is selected from activin-A (ACT), retinoic acid (RA), epidermal growth factor, bone morphogenetic protein, TGF-β, hepatocyte growth factor, platelet-derived growth factor, TGF-α, IGF-I and II, hematopoietic growth factors, heparin binding growth factor, peptide growth factors, erythropoietin, interleukins, tumor necrosis factors, interferons, colony stimulating factors, fibroblast growth factors, nerve growth factor (NGF) and muscle morphogenic factor (MMF).

69. The method of claim 60, wherein the predetermined period of time is two weeks.

70. The method of claim 60, wherein culturing is conducted in a serum-free medium.
